(19) United States
(12) Patent Application Publication
(10) Pub. No.: US 2025/0186474 A1
(43) Pub. Date: Jun. 12, 2025

(54) PHARMACEUTICAL SOLUTION COMPOSITION AND ORAL MIST INHALER COMPRISING THE SAME

(71) Applicant: CHINA CHEMICAL & PHARMACEUTICAL CO., LTD., TAIPEI CITY (TW)

(72) Inventors: CHI-LI KUO, TAIPEI CITY (TW); SHAO-MING LU, TAIPEI CITY (TW); PAO-LIN LIAO, TAIPEI CITY (TW); CHIA-HUI WU, TAIPEI CITY (TW)

(21) Appl. No.: 18/651,022

(22) Filed: Apr. 30, 2024

(30) Foreign Application Priority Data
Dec. 7, 2023 (TW) 112147700

(51) Int. Cl. A61K 31/7068; A61K 9/00; A61K 9/08

(52) U.S. Cl. CPC A61K 31/7068; A61K 9/0078; A61K 9/08

(57) ABSTRACT

A pharmaceutical solution composition includes: a therapeutically effective amount of molnupiravir or a metabolite thereof, and a solvent. An oral mist inhaler includes: a soft mist inhaler and the pharmaceutical solution composition, wherein the soft mist inhaler is loaded with the pharmaceutical solution composition. The pharmaceutical solution composition is highly stable and produces a relatively small amount of impurities. The oral mist inhaler allows a user to inhale the pharmaceutical solution composition directly so that not only can the composition be rapidly absorbed, but also the metabolic process that the drug must go through is reduced to enhance the bioavailability of the drug. The particles formed by the soft mist inhaler atomizing the composition are small and sprayed in a large amount. Compared with the conventional oral administration method, the oral mist inhaler is easy to use and helps improve the comfort and convenience of medicine consumption.

### BACKGROUND OF THE INVENTION

### 1. Technical Field

The present invention relates to a pharmaceutical solution composition and more particularly to a pharmaceutical solution composition for treating viral infection. The invention also relates to an oral mist inhaler containing the pharmaceutical solution composition and to applications of the oral mist inhaler.

### 2. Description of Related Art

As one of the major causes of human diseases, viral infection poses a serious threat to global public health and impacts social stability and economic development. A virus is a submicroscopic infectious agent and depends entirely on the energy and metabolic systems of a host cell to obtain the substances for replication activities.

The coronavirus disease 2019 (COVID-19) caused by severe acute respiratory syndrome coronavirus 2 (SARS-CoV-2) broke out at the end of 2019. As the virus was highly pathogenic, and the disease highly contagious, spreading across mainland China within a noticeably short time, the World Health Organization classified COVID-19 as an epidemic disease, and COVID-19 has changed the lives of people around the world drastically since its outbreak.

The most common symptoms of COVID-19 include fever, dry coughs, and difficulty in breathing. COVID-19 may also affect the gastrointestinal system, the liver, the cardiovascular system, the kidneys, the nervous system, and other organs. Considering the extremely high morbidity rate and mortality rate of COVID-19, scientists have been actively studying a wide range of drugs, vaccines, and biological products in order to find ways to treat diseases caused by the virus.

Of all the antiviral drugs that can be used to treat COVID-19, the first choices are such active pharmaceutical ingredients (APIs) as Favipiravir, Remdesivir, Molnupiravir, and GS-441524. Molnupiravir (MK-4482 or EIDD-2801) is an orally active antiviral drug for treating hepatitis and influenza in the first place. EIDD-2801, in particular, is an isopropyl prodrug of N4-hydroxycytidine (which is a synthetic nucleoside derivative), has the molecular formula C₁₃H₁₉N₃O₇ and a molecular weight of 329.31 g·mol⁻¹, and can be hydrolyzed in vivo to produce the intermediate EIDD-1931 (β-D-N4-hydroxycytidine, or NHC), which is distributed into cells and forms pharmaceutically active EIDD-1931-5′-triphosphate (or NHC-TP) through phosphorylation by a host kinase. NHC-TP can bind to the SARS-CoV-2 RNA by the action of a virus RNA polymerase, i.e., nsp12, to cause genomic error accumulation and thereby inhibit viral replication, and this process is referred to as a viral error catastrophe.

### BRIEF SUMMARY OF THE INVENTION

This section of the specification aims to provide a simplified version of the contents of the present invention so that a reader can have a basic understanding of the invention. This section, therefore, is neither an exhaustive description of the invention nor intended to point out all the important/key elements of feasible embodiments of the invention or define the scope of the invention.

As a medicine for treating COVID-19, molnupiravir is administered to patients typically through the mouth. Oral administration, however, may reduce the intake of the API by a host of the virus, and this is because orally administered molnupiravir must be absorbed by the gastrointestinal system in order to be delivered to the blood and produce metabolites of molnupiravir and get into the cells of host.

In view of the demand for treating diseases caused by SARS-CoV-2, the present invention provides a composition that includes molnupiravir or a metabolite thereof. The composition can be atomized so that an individual can directly inhale the composition either orally or nasally to reduce the metabolic process required by oral administration, thereby enhancing the bioavailability of the drug in the individual's body.

According to one aspect of the present invention, a pharmaceutical solution composition includes a therapeutically effective amount of molnupiravir or a metabolite thereof, and a solvent.

In one embodiment of the present invention, the metabolite of molnupiravir is EIDD-1931 (β-D-N4-hydroxycytidine).

In one embodiment of the present invention, the solvent is water.

In one embodiment of the present invention, the pharmaceutical solution composition further includes a pharmaceutically acceptable excipient.

In one embodiment of the present invention, the excipient is a preservative or a chelating agent.

In one embodiment of the present invention, the preservative is benzalkonium chloride (BKC), benzethonium chloride, benzododecinium bromide, benzoic acid, benzyl alcohol, butylparaben, cetylpyridinium chloride (CPC), metacresol, methylparaben (MP), phenol, potassium sorbate, propylparaben, sodium borate, sorbic acid, or thimerosal, and the chelating agent is ethylenediaminetetraacetic acid (EDTA), edetate calcium disodium, anhydrous edetate calcium disodium, edetate disodium (EDTA-2Na), gluceptate sodium, or pentetic acid or a salt thereof.

In one embodiment of the present invention, the molnupiravir or the metabolite thereof has a concentration of 2 mg/ml, 5 mg/ml, or 10 mg/ml.

In one embodiment of the present invention, the pharmaceutical solution composition is administered by way of nasal drops, a nebulizer for oral/nasal inhalation, a nasal spray, a soft mist inhaler (SMI) for oral inhalation, or a metered-dose inhaler (MDI) for oral inhalation.

According to another aspect of the present invention, an oral mist inhaler includes a soft mist inhaler and the foregoing pharmaceutical solution composition, wherein the soft mist inhaler is loaded with the pharmaceutical solution composition.

In one embodiment of the present invention, the pharmaceutical solution composition is atomized by the soft mist inhaler to form atomized particles, and the average sprayed amount of the atomized particles per spray is at least 13 mg.

In one embodiment of the present invention, the pharmaceutical solution composition is atomized by the soft mist inhaler to form atomized particles, and at least 50% of the atomized particles are smaller than 5.8 μm.

In one embodiment of the present invention, at least 60% of the atomized particles are smaller than 5.8 μm.

The present invention has the following advantages: The pharmaceutical solution composition of the invention is highly stable and produces a relatively small amount of impurities. In addition, the pharmaceutical solution composition can be used in an oral mist inhaler so that a user can inhale the composition through the oral mist inhaler into the lungs. As the lungs are covered with capillaries, this administration method not only allows the composition to be rapidly absorbed, but also reduces the metabolic process the active pharmaceutical ingredient must go through in the user's body, thereby increasing the bioavailability of the drug significantly. Moreover, the particles formed by atomizing the composition are small and sprayed in an amount of at least 13 mg per spray. Compared with the conventional oral administration method, the oral mist inhaler of the invention is easier to use and can enhance the comfort and convenience of medicine consumption.

### BRIEF DESCRIPTION OF THE SEVERAL VIEWS OF THE DRAWINGS

The aforesaid and other objectives, features, advantages, and embodiments of the present invention can be better understood by referring to the following detailed description in conjunction with the accompanying drawings.

FIG. 1A and FIG. 1B are bar charts showing stress test results of the pharmaceutical solution composition of the present invention, with FIG. 1A showing the results in terms of single-impurity percentage, and FIG. 1B in terms of total-impurity percentage.

FIG. 2A to FIG. 2C are graphs showing the results of a particle size distribution test performed on the pharmaceutical solution composition of the present invention in triplicate.

FIG. 3 shows the results of a plaque assay performed on the pharmaceutical solution composition of the present invention in triplicate.

FIG. 4A to FIG. 4I are images of biopsy sections of the lung tissue of non-virus-infected hamsters, showing in vivo test results regarding the effect of the pharmaceutical solution composition of the present invention on the lung tissue, with FIG. 4A to FIG. 4C showing the results of the group that was neither infected with the virus nor given medication (n=3), and FIG. 4D to FIG. 4I showing the results of the group that was not infected with the virus but was given the pharmaceutical solution composition (n=6), wherein: the arrow in FIG. 4D indicates osseous metaplasia in alveoli, the arrow and the circles in FIG. 4E indicate osseous metaplasia in alveoli and extravasated blood cells in alveolar lumina respectively, and the arrow in FIG. 4H indicates osseous metaplasia in alveoli.

FIG. 5A to FIG. 5L are images of biopsy sections of the lung tissue of non-virus-infected and virus-infected hamsters, showing in vivo test results regarding the effects of different doses of the pharmaceutical solution composition of the present invention on the lung tissue. More specifically, FIG. 5A to FIG. 5C show the results of the group that was neither infected with the virus nor given medication (n=6), wherein the arrow in FIG. 5C indicates interstitial inflammation with foreign body; FIG. 5D to FIG. 5F show the results of the group that was infected with the virus but was not given medication (n=6), wherein: the arrows in FIG. 5D indicate vascular endothelialitis, the * symbol in FIG. 5D indicates bronchiolar epithelial cell degeneration/necrosis and cell inflammation, the arrow in FIG. 5E indicates bronchiolar epithelial cell degeneration/necrosis, the * symbols in FIG. 5E indicate cell inflammation, and the circle in FIG. 5F indicates bronchial epithelial cell degeneration/necrosis; FIG. 5G to FIG. 5I show the results of the group that was infected with the virus and was given the pharmaceutical solution composition in a low dose (1 mg/ml) (n=6), wherein: the arrows in FIG. 5G indicate bronchiolar epithelial cell degeneration/necrosis, the * symbols in FIG. 5G indicate cell inflammation, the circle in FIG. 5H indicates bronchiolitis, and the * symbols in FIG. 5H indicate cell debris and heterophils within the lumen; and FIG. 5J to FIG. 5L show the results of the group that was infected with the virus and was given the pharmaceutical solution composition in a high dose (5 mg/ml) (n=6), wherein: the arrows in FIG. 5J indicate interstitial mixed cell infiltration, the arrow in FIG. 5K indicates interstitial mixed cell infiltration, the * symbol in FIG. 5K indicates peribronchial mixed cell infiltration, the arrow in FIG. 5L indicates bronchiolar epithelial cell degeneration/necrosis, and the * symbols in FIG. 5L indicate cell inflammation.

### DETAILED DESCRIPTION OF THE INVENTION

The technical contents of the present invention are detailed below with reference to the accompanying drawings, which, for the ease of illustration, are not necessarily drawn to scale. The drawings and the proportions illustrated therein are not intended to be restrictive of the scope of the invention.

Unless otherwise defined, all the technical and scientific terms used herein have the same connotations as understood by a person of ordinary skill in the art. Moreover, as used throughout this specification, the terms defined in the following paragraph should be construed as defined.

The term “or” means “and/or” unless otherwise stated. The term “include” does not exclude the addition of one or more components, steps, operations, or elements to the component(s), step(s), operation(s), or element(s) following the term. The terms “comprise,” “include,” “contain,” “encompass,” and “have” as used herein are interchangeable and are non-limiting. The articles “a” and “the” when used together with a singular object may connote referral to a plurality of such objects, unless otherwise specified in the context. For example, the terms “a,” “the,” “one or more,” and “at least one” are interchangeable herein.

One aspect of the present invention provides a pharmaceutical solution composition that includes: a therapeutically effective amount of molnupiravir or a metabolite thereof, and a solvent. In a preferred embodiment, the metabolite of molnupiravir is EIDD-1931 (β-D-N4-hydroxycytidine). According to an embodiment of the invention, the solvent is water, glycerol, propylene glycol, polyethylene glycol, polypropylene glycol, ethanol, isopropyl alcohol, a mineral oil, or peanut oil. In a preferred embodiment, the solvent is water.

According to an embodiment of the present invention, the pharmaceutical solution composition further includes a pharmaceutically acceptable excipient. In a preferred embodiment, the excipient is a preservative or a chelating agent. More specifically, the preservative is benzalkonium chloride (BKC), benzethonium chloride, benzododecinium bromide, benzoic acid, benzyl alcohol, butylparaben, cetylpyridinium chloride (CPC), metacresol, methylparaben (MP), phenol, potassium sorbate, propylparaben, sodium borate, sorbic acid, or thimerosal. In a preferred embodiment, the preservative is benzalkonium chloride (BKC). The chelating agent is ethylenediaminetetraacetic acid (EDTA), edetate calcium disodium, anhydrous edetate calcium disodium, edetate disodium (EDTA-2Na), gluceptate sodium, or pentetic acid or a salt thereof. In a preferred embodiment, the chelating agent is ethylenediaminetetraacetic acid (EDTA).

The inclusion of molnupiravir or a metabolite thereof and the addition of a solvent and an excipient enable the pharmaceutical solution composition of the present invention to treat diseases caused by a virus such as a coronavirus (e.g., SARS-CoV-1, which causes the severe acute respiratory syndrome, or SARS; MERS-COV, which causes the Middle East respiratory syndrome, or MERS; or SARS-CoV-2, which causes COVID-19), a norovirus, the Chikungunya virus, the Ebola virus, an influenza virus, a syncytial virus, the Venezuelan equine encephalitis virus (VEEV), a diarrhea virus, or the hepatitis C virus. In a preferred embodiment, the pharmaceutical solution composition of the invention is used to treat COVID-19.

According to an embodiment of the present invention, the concentration of the molnupiravir or the metabolite thereof is 2-20 mg/ml, such as but not limited to 2 mg/ml, 3 mg/ml, 4 mg/ml, 5 mg/ml, 6 mg/ml, 7 mg/ml, 8 mg/ml, 9 mg/ml, 10 mg/ml, 11 mg/ml, 12 mg/ml, 13 mg/ml, 14 mg/ml, 15 mg/ml, 16 mg/ml, 17 mg/ml, 18 mg/ml, 19 mg/ml, or 20 mg/ml. In a preferred embodiment, the concentration of the molnupiravir or the metabolite thereof is 2 mg/ml, 5 mg/ml, or 10 mg/ml. The inventor of the present invention has found through a series of experiments that the maximum solubility of the concentration of the molnupiravir or the metabolite thereof in the pharmaceutical solution composition is 20 mg/ml, and that when the concentration of the molnupiravir or the metabolite thereof is 2 mg/ml, 5 mg/ml, or 10 mg/ml, the pharmaceutical solution composition has the highest drug stability and does not form drug precipitates.

According to an embodiment of the present invention, the pharmaceutical solution composition is administered by way of nasal drops, a nebulizer for oral/nasal inhalation, a nasal spray, a soft mist inhaler (SMI) for oral inhalation, or a metered-dose inhaler (MDI) for oral inhalation. The pharmaceutical solution composition of the invention is administered to an individual not by the conventional oral administration method but by spraying or atomization so that, when inhaled into the lungs through an inhaler, the small molecules of the composition can be rapidly absorbed thanks to the large contact area provided by, and the capillaries covering, the lungs, and this enhances drug absorption significantly. Moreover, the pharmaceutical solution composition can be administered nasally as well as orally, and this provides an alternative drug administration method for those who have difficulty in swallowing or fear swallowing drugs, making it more convenient to take medicine.

Another aspect of the present invention provides an oral mist inhaler that includes a soft mist inhaler and the foregoing pharmaceutical solution composition, wherein the soft mist inhaler is loaded with the pharmaceutical solution composition. The oral mist inhaler can atomize the pharmaceutical solution composition and thereby turn the composition into small molecules to facilitate absorption. More specifically, the oral mist inhaler allows a user to inhale the composition in the form of minute particles directly through the mouth. Compared with the conventional oral administration method, the oral mist inhaler of the invention reduces the metabolic process that molnupiravir must go through in the user's body, and this enhances the absorption and bioavailability of active pharmaceutical ingredient. The oral mist inhaler also helps improve the comfort and convenience of medicine consumption. In addition, the oral mist inhaler is easy to use and is suitable for patients of all ages.

According to an embodiment of the present invention, the pharmaceutical solution composition is atomized by the soft mist inhaler to form atomized particles, and the average sprayed amount of the atomized particles per spray is at least 13 mg, such as but not limited to at least 13 mg, at least 14 mg, at least 15 mg, at least 16 mg, at least 17 mg, at least 18 mg, at least 19 mg, at least 20 mg, at least 21 mg, at least 22 mg, at least 23 mg, at least 24 mg, or at least 25 mg.

According to an embodiment of the present invention, the pharmaceutical solution composition is atomized by the soft mist inhaler to form atomized particles, in which the percentage of atomized particles smaller than 5.8 μm is at least 50%, such as but not limited to at least 50%, at least 55%, at least 60%, at least 65%, at least 70%, at least 75%, at least 80%, at least 85%, at least 90%, at least 95%, or at least 99%. In a preferred embodiment, atomized particles smaller than 5.8 μm constitute at least 60% of all the atomized particles. The oral mist inhaler of the invention has been experimentally proved to be able to provide a sufficient sprayed amount per spray, so a patient using the oral mist inhaler to administer the pharmaceutical solution composition will not experience inconsistent drug administration caused by an insufficient sprayed amount. Furthermore, with at least 60% of the atomized particles of the pharmaceutical solution composition being smaller than 5.8 μm, it is obvious that the small molecule/particle sizes of the composition will make it easy for the drug to enter respiratory system of host and be better absorbed by patients.

### EMBODIMENTS

It should be understood that the examples and embodiments described herein serve only to expound the present invention and provide those skilled in the art with suggestions on making various modifications or changes based on the examples and embodiments. All such modifications and changes shall be viewed as encompassed by the spirit and scope of the invention and falling with the scope of the appended claims. All the publications, patents, and published patent applications cited herein, if any, are incorporated into this specification in their entirety by reference for all purposes.

### 1. Preparation of a Pharmaceutical Solution Composition

In this test, a pharmaceutical solution composition (hereinafter referred to as the composition for short) was prepared from an effective amount of an active pharmaceutical ingredient, a solvent, a preservative, and a chelating agent. The formula of the composition is shown in Table 1.

TABLE 1
Formula and stability test results of a pharmaceutical solution composition

| Composition/ | R01-1 | R01-2 | R01-3 | R01-4 | R01-5 | R07 | R08 | R09 |
|---|---|---|---|---|---|---|---|---|
| pH value | (water) | (pH 3) | (pH 4) | (pH 5) | (pH 6) | (pH 7) | (pH 8) | (pH 9) |
| Unit | mg/ml | mg/ml | mg/ml | mg/ml | mg/ml | mg/ml | mg/ml | mg/ml |
| EIDD-1931 | 20 | 20 | 20 | 20 | 20 | 20 | 20 | 20 |
| Anhydrous | — | 16.43 | 11.99 | 7.03 | 2.42 | — | — | — |
| citric acid | | | | | | | | |
| Trisodium | — | 4.27 | 11.05 | 18.64 | 25.7 | — | — | — |
| citrate | | | | | | | | |
| dihydrate | | | | | | | | |
| Tris | — | — | — | — | — | 12.1 | 12.1 | 12.1 |
| Hydrogen | — | — | — | — | — | Proper | Proper | Proper |
| chloride | | | | | | amount to | amount to | amount to |
| | | | | | | reach pH 7 | reach pH 8 | reach pH 9 |
| BKC | 0.1 | 0.1 | 0.1 | 0.1 | 0.1 | 0.1 | 0.1 | 0.1 |
| EDTA | 0.1 | 0.1 | 0.1 | 0.1 | 0.1 | 0.1 | 0.1 | 0.1 |
| Water | Proper | Proper | Proper | Proper | Proper | Proper | Proper | Proper |
| | amount to | amount to | amount to | amount to | amount to | amount to | amount to | amount to |
| | reach 1 ml | reach 1 ml | reach 1 ml | reach 1 ml | reach 1 ml | reach 1 ml | reach 1 ml | reach 1 ml |
| Stress test | Theoretical | Theoretical | Theoretical | Theoretical | Theoretical | Theoretical | Theoretical | Theoretical |
| | value/ | value/ | value/ | value/ | value/ | value/ | value/ | value/ |
| | single- | single- | single- | single- | single- | single- | single- | single- |
| | impurity | impurity | impurity | impurity | impurity | impurity | impurity | impurity |
| | percentage/ | percentage/ | percentage/ | percentage/ | percentage/ | percentage/ | percentage/ | percentage/ |
| | total- | total- | total- | total- | total- | total- | total- | total- |
| | impurity | impurity | impurity | impurity | impurity | impurity | impurity | impurity |
| | percentage | percentage | percentage | percentage | percentage | percentage | percentage | percentage |
| 60° C., day 0 | 102.72/ | 101.76/ | 101.15/ | 80.07/ | 89.18/ | 101.41/ | 103.15/ | 99.44/ |
| | 0.05/ | 0.06/ | 0.09/ | 0.09/ | 0.06/ | 0.02/ | 0.02/ | 0.05/ |
| | 0.05 | 0.06 | 0.12 | 0.09 | 0.06 | 0.06 | 0.08 | 0.13 |
| 60° C., day 7 | 100.93/ | 87.79/ | 97.55/ | 100.95/ | 103.55/ | 101.46/ | 103.45/ | 100.09/ |
| | 0.07/ | 14.42/ | 2.94/ | 0.48/ | 0.17/ | 0.18/ | 0.28/ | 0.80/ |
| | 0.13 | 15.02 | 3.14 | 0.55 | 0.28 | 0.51 | 1.20 | 3.01 |

### 1-1. pH Stability

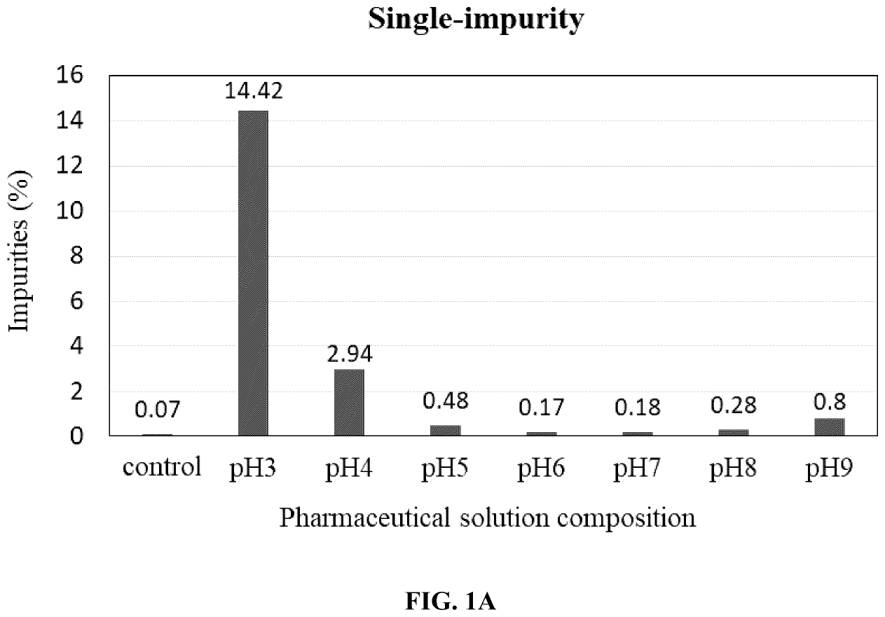

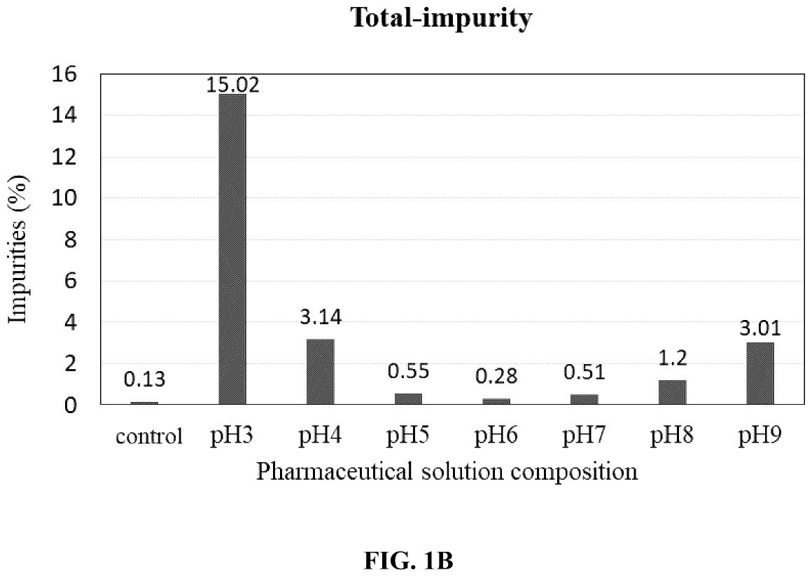

Referring to FIG. 1, the stability of the composition at different pH values was tested, with pH3-6 achieved with citric acid buffers, and pH7-9 with a Tris buffer. The stability test included a stress test conducted at 60° C. for seven days. By comparing the test result of each group on day 7 with the result of the group on day 0 (as the corresponding control-group result), it was found that group R01-1 (without pH adjustment) was the most stable and produced the least impurities (with the single-impurity percentage being 0.07, and the total-impurity percentage being 0.13).

### 1-2. Sprayed Amount Test

It was known from the pH stability test that the R01-1 composition was the most stable and produced the least impurities. Therefore, the R01-1 composition was used in the sprayed amount test. The sprayed amount test included spraying the R01-1 composition with Mei Rui Zi Inhaler M-100 (non-sterile) and recording the sprayed amount of each spray. A pre-test was performed before the formal test. The pre-test included spraying for five times, and the formal sprayed amount test included spraying for 55 times. The test results are shown in Table 2.

TABLE 2
Sprayed amount test

| Pre-test | | Formal test | | | | | |
|---|---|---|---|---|---|---|---|
| | Sprayed | | Sprayed | | Sprayed | | Sprayed |
| No. of | amount | No. of | amount | No. of | amount | No. of | amount |
| spray | (mg) | spray | (mg) | spray | (mg) | spray | (mg) |
| 1 | 11.4 | 6 | 14.3 | 24 | 13.1 | 42 | 13 |
| 2 | 12.1 | 7 | 13.7 | 25 | 13.7 | 43 | 13.7 |
| 3 | 15.1 | 8 | 13.6 | 26 | 14.5 | 44 | 12.3 |
| 4 | 14.8 | 9 | 13.4 | 27 | 15.2 | 45 | 12.8 |
| 5 | 12.7 | 10 | 13.9 | 28 | 14.8 | 46 | 13.7 |

| — | 11 | 13.8 | 29 | 14.3 | 47 | 13.4 |
|---|---|---|---|---|---|---|
| | 12 | 13.3 | 30 | 13.9 | 48 | 12.3 |
| | 13 | 13.2 | 31 | 13.5 | 49 | 13.1 |
| | 14 | 13.3 | 32 | 13.6 | 50 | 13.1 |
| | 15 | 13.1 | 33 | 13.8 | 51 | 13.8 |
| | 16 | 13.5 | 34 | 13 | 52 | 13.8 |
| | 17 | 13.1 | 35 | 13.4 | 53 | 12.8 |
| | 18 | 13.9 | 36 | 13 | 54 | 13.2 |
| | 19 | 13.1 | 37 | 13.9 | 55 | 13.7 |
| | 20 | 13 | 38 | 14.3 | 56 | 13.3 |
| | 21 | 13.1 | 39 | 14 | 57 | 13.7 |
| | 22 | 13.5 | 40 | 12.3 | 58 | 13.2 |
| | 23 | 13.3 | 41 | 13.4 | 59 | 13.4 |
| | | | | | 60 | 13.9 |
| | Average | 13.5 | Standard | 0.6 | Relative | 4.1 |
| | | | deviation | | standard | |
| | | | (SD) | | deviation | |
| | | | | | (RSD) | |

### 1-3. Particle Size Distribution (PSD) Test

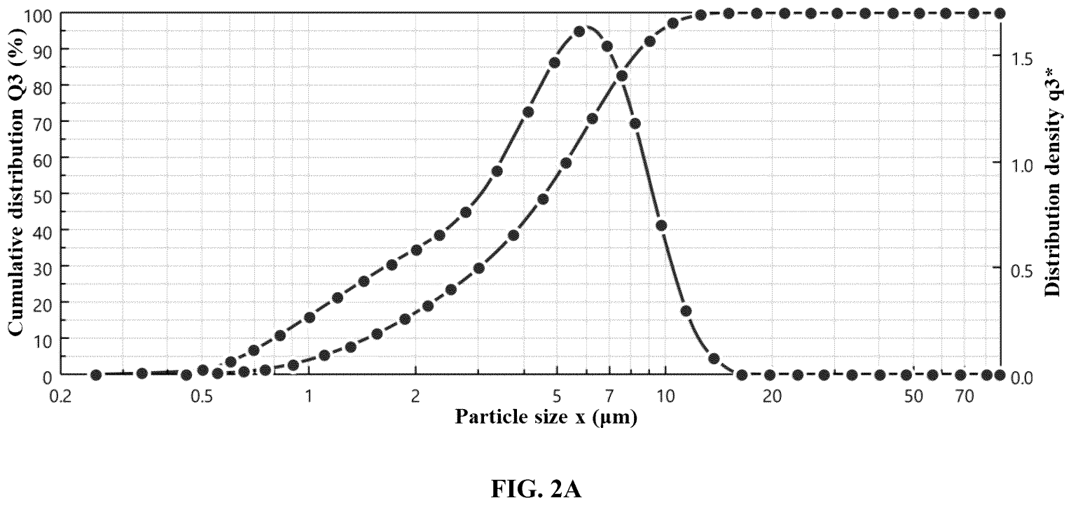

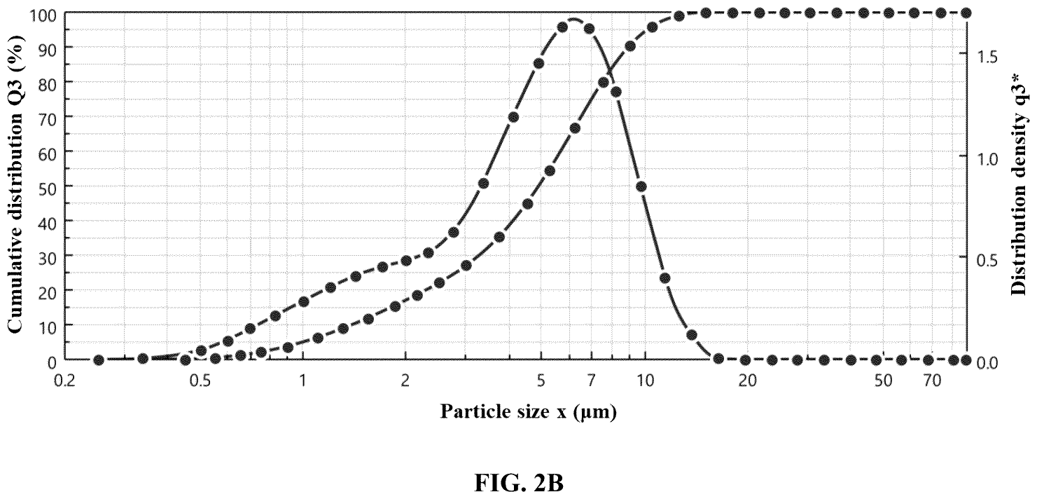

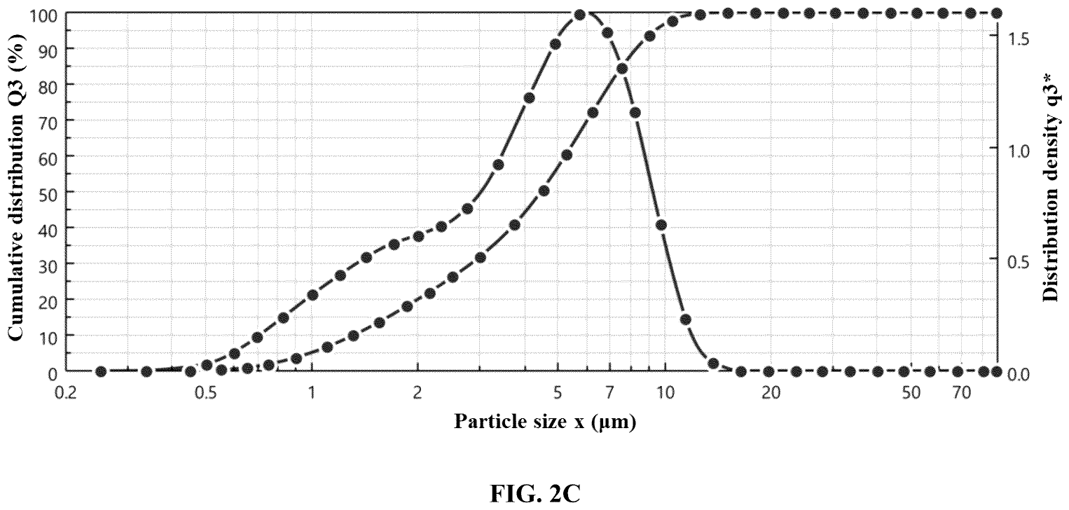

Reference is made to FIG. 2. The particle size distribution test was performed to determine the size distribution of the atomized particles of the composition. The test included preparing a 1000 ml solution of the R01-1 composition and analyzing particle size distribution with a laser particle size analyzer. The test was conducted in triplicate, and the results are shown in Table 3 and FIG. 2.

TABLE 3
Particle size distribution test results

| R01-1 | x_{10, 3} | x_{50, 3} | x_{90, 3} | Cₒₚₜ | Q₃ (x = 5.8 |
|---|---|---|---|---|---|
| composition | (μm) | (μm) | (μm) | (%) | μm) (%) |
| #1 | 1.46 | 4.61 | 8.63 | 10.35 | 65.20 |
| #2 | 1.39 | 4.89 | 8.96 | 9.33 | 61.45 |
| #3 | 1.31 | 4.45 | 8.42 | 9.17 | 67.02 |

According to the above, the average sprayed amount per spray was 13.5 mg±0.6 mg (see the formal test results in Table 2), and the percentage of average effective particles (i.e., atomized particles smaller than 5.8 μm) was 64.55% (see Table 3 and FIG. 2). The test results indicate that the particles formed by atomizing the pharmaceutical solution composition of the present invention had small particle sizes, and that the sprayed amount per spray was at least 13 mg.

### 1-4. Accelerated Stability Test

An accelerated stability test was performed to test the 0-month, 1-month, 3-month, and 6-month stability of the R01-1 composition at 40° C. The test results are shown in Table 4.

TABLE 4
Accelerated stability test results

| | |
|---|---|
| Composition | R01-1 (water) |
| Unit | mg/ml |
| EIDD-1931 | 20 |
| BKC | 0.1 |
| EDTA | 0.1 |
| Water | Proper amount to reach 1 ml |

| | Theoretical | Single-impurity percentage/ |
|---|---|---|
| Stress test | value (%) | total-impurity percentage (%) |
| 0 M | 102.72 | 0.05/0.05 |
| 40° C., 1 M | 101.86 | 0.07/0.13 |
| 40° C., 3 M | 101.61 | 0.20/0.36 |
| 40° C., 6 M | 102.41 | 0.49/0.89 |

The 40° C. 6-month stability test results show that the percentage of the impurity uridine in the R01-1 composition increased to 0.49%. As uridine is an endogenous substance in the human body and exists in an adult's body and a child's body at 3-8 μM and 0.5-5 μM respectively, the concentration of the impurity uridine in a human body is calculated with the following formula:

$$\frac{\begin{matrix}dose of composition ( 10 mg / ml ) \times volume of each spray ( 0.0135 ml ) \times \\ uridine concentration ( 0.49 \% ) \times number of sprays ( 2 )\end{matrix}}{molecular weight of uridine ( 244.2 ) \times volume of blood in human body ( 5 L )} = 0.00108 μM$$

The calculation result indicates that the impurity uridine in the composition of the present invention had an equivalent concentration of 0.00108 μM in the 6-month stability test, wherein 0.00108 μM is about 0.216% of the blood uridine concentration of a child (0.5 μM). Therefore, the upper limit of the percentage of the impurity uridine was set at 2%; that is to say, as far as a human body is concerned, the acceptable amount of the impurity produced is 2% or less.

### 1-5. Optimal Formula of the Pharmaceutical Solution Composition

The foregoing test results show that a drug prepared with 20 mg/ml EIDD-1931 had precipitation issues. Therefore, the concentration of EIDD-1931 was adjusted, and another stress test was conducted to evaluate how the resulting drugs degrade in 7 and 14 days. The test results are shown in Table 5.

TABLE 5
Pharmaceutical solution compositions with different EIDD-1931 concentrations

| Group | R11 | | R12 | | R13 | |
|---|---|---|---|---|---|---|
| Unit | mg/ml | | mg/ml | | mg/ml | |
| EIDD-1931 | 10 | | 5 | | 2 | |
| BKC | 0.1 | | 0.1 | | 0.1 | |
| EDTA | 0.1 | | 0.1 | | 0.1 | |
| Water | Proper amount to reach 1 ml | | Proper amount to reach 1 ml | | Proper amount to reach 1 ml | |
| | | Single- | | Single- | | Single- |
| | | impurity | | impurity | | impurity |
| | | percentage/ | | percentage/ | | percentage/ |
| | | total- | | total- | | total- |
| | | impurity | | impurity | | impurity |
| Impurity | Theoretical | percentage | Theoretical | percentage | Theoretical | percentage |
| test | value (%) | (%) | value (%) | (%) | value (%) | (%) |
| API | 93.25 | 0.01/0.01 | | | | |
| 60° C., day 0 | 100.87 | 0.01/0.01 | 106.09 | 0.01/0.01 | 102.72 | 0.01/0.01 |
| 60° C., day 7 | 102.32 | 0.09/0.13 | 106.18 | 0.09/0.13 | 103.06 | 0.09/0.12 |
| 60° C., day 14 | 102.02 | 0.24/0.31 | 104.18 | 0.23/0.34 | 102.77 | 0.23/0.31 |

According to the test results, the percentage of the impurity in each drug increased after the drug was stressed at 60° C. for 7 days and 14 days, but the amounts of the impurity produced were still within the acceptable range (2%). In the following animal test, therefore, the compositions with the EIDD-1931 concentrations of 2 mg/ml, 5 mg/ml, and 10 mg/ml were used.

After analyzing the stability and optimal formula of the pharmaceutical solution composition, an animal test was performed to test the effects of the composition in an animal's body. The animal test began after an in vitro plaque assay, which tested the antiviral effect of the composition. After that, the pharmacokinetics (PK) of nasal administration of the composition to hamsters was studied by measuring the EIDD-1931 concentration of the hamster's blood plasma. Lastly, an animal challenge study was conducted. The hamsters were sacrificed three days after viral infection, and lung tissue was taken from the hamsters and sectioned for examination in order to know how the effect of composition in the lungs.

### 2. Viral Plaque Assay

The viral plaque assay was performed to test the antiviral effect of the pharmaceutical solution composition of the present invention against SARS-CoV-2. The viral strain used in the assay was SARS-CoV-2 Omicron BA.5. The infected cells were Vero E6 cells. The concentrations of the composition were 0.3 μM, 1.5 μM, and 3 μM. The assay included a solvent control group and a virus control group.

The assay was conducted as follows. A 24-well plate was inoculated with Vero E6 cells at a concentration of 2×10⁵ cells per well, and each well was added with a Dulbecco's modified Eagle medium (DMEM) containing 10% fetal bovine serum (FBS), 100 units/ml penicillin G sodium, 100 μg/ml streptomycin sulfate, and 250 ng/ml amphotericin B. The cells were left in the culture medium for one day. On the next day, i.e., the day of viral infection, each group of cells was treated with the pharmaceutical solution composition of a specified concentration for one hour, and then the composition was removed. Next, the cells were infected with SARS-CoV-2 Omicron BA.5 for one hour. Following that, the cell culture medium was removed, and the cells were washed with phosphate buffered saline (PBS) once and then cultured for five days in a liquid culture medium containing 1% methyl cellulose. Afterward, the cells were fixated with 10% formaldehyde for one hour, and the culture medium was subsequently removed. The cells were then stained with 0.5% crystal violet, and the number of plaques was recorded.

The viral inhibition rate was calculated using the formula

$$[ 1 - ( \frac{VD}{VC} ) ] \times 100 \% ,$$

where VD and VC represent the number of plaques in the presence of a composition under test and the number of plaques in the presence of the corresponding solvent, respectively.

The half maximal effective concentration (EC₅₀) of each composition under test was obtained using the formula

$$ConcH - [ \frac{( ConH \% - 50 \% ) ( ConcH - ConcL )}{ConcH \% - ConcL \%} ] ,$$

where: ConcH represents the concentration that inhibited 50% or more of the viruses, and ConcL represents the concentration that inhibited less than 50% of the viruses.

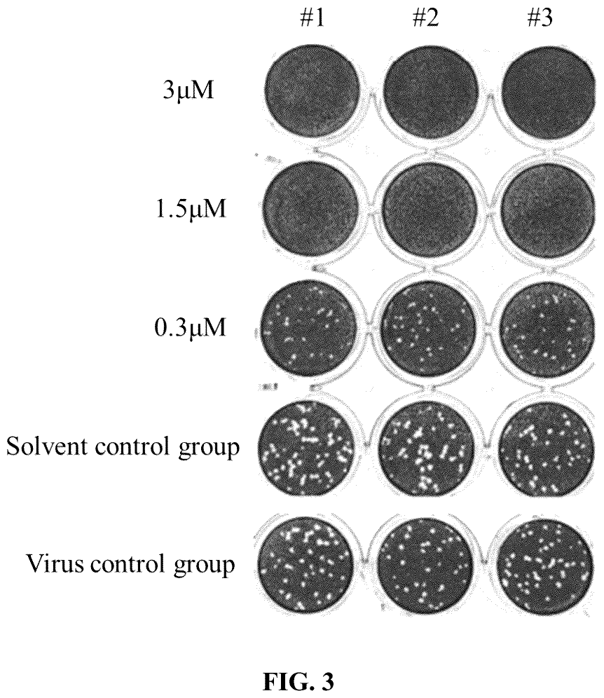

The assay results are shown in FIG. 3 and Table 6. Compared with the solvent control group, the 1.5 μM and 3 μM pharmaceutical solution compositions had 100% viral inhibition rates, with the EC₅₀ being 0.543±0.065 μM.

TABLE 6
Viral inhibition rate

| | | Viral inhibition rate (%) | | | | |
|---|---|---|---|---|---|---|
| | | | | | | Standard |
| Concentration | | #1 | #2 | #3 | Average | deviation |
| 3 | μM | 100% | 100% | 100% | 100% | 0% |
| 1.5 | μM | 100% | 100% | 100% | 100% | 0% |
| 0.3 | μM | 34% | 34% | 43% | 37% | 4% |

### 3. Pharmacokinetic Test

The pharmacokinetic test was performed to verify whether nasal administration (or nasal dispensing to be exact) enables the main ingredient (EIDD-1931) to be detected in blood. The animal model used in the test was hamsters, and the hamsters were divided into the following groups:
1. Group without viral infection or medication (n=3)
2. Group without viral infection but with 5 mg/ml EIDD-1931 (n=6)

The drug, i.e., 5 mg/ml EIDD-1931, was given at a dose of 50 μl by nasal dispensing, and blood was collected from the hamsters before drug administration (in the 0^{th} hour) and in the 1^{st} hour and 24^{th} hour after drug administration while the hamsters were being raised. 24 hours after drug administration, the hamsters were sacrificed, and blood and lung tissue were collected. The blood specimens were analyzed by liquid chromatography with tandem mass spectrometry (LC-MS/MS) to determine the concentration of EIDD-1931 in the hamster plasma. The following animal challenge study was conducted only on hamsters whose 24^{th}-hour blood test results showed that the main ingredient in the blood had reached the effective concentration (1 μM).

The lung tissue was fixated and preserved in paraformaldehyde. To facilitate observation under a microscope, the lung tissue was further trimmed, treated, embedded in paraffin, cut into about 5 μm-thick slices, and stained with hematoxylin and eosin.

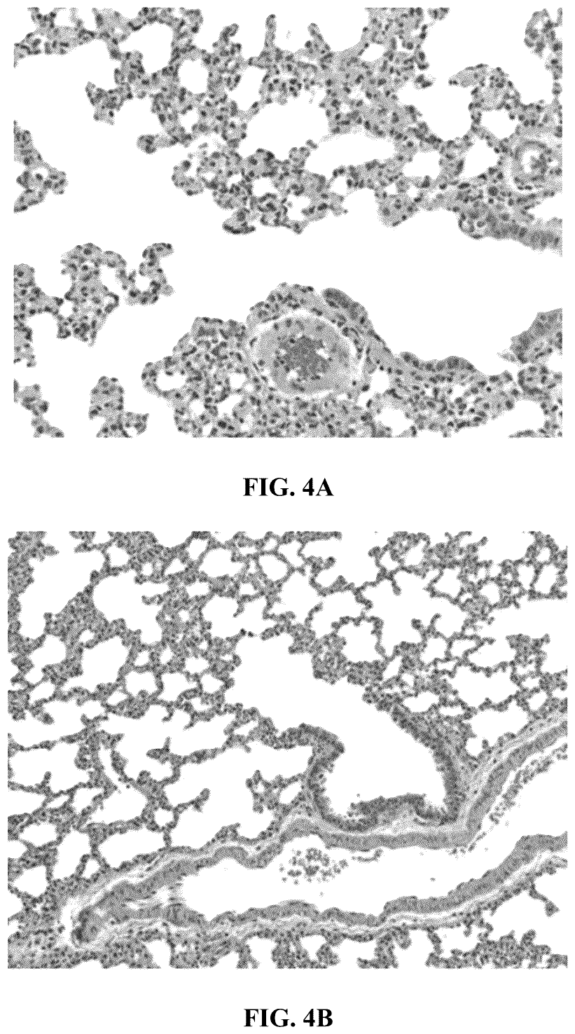

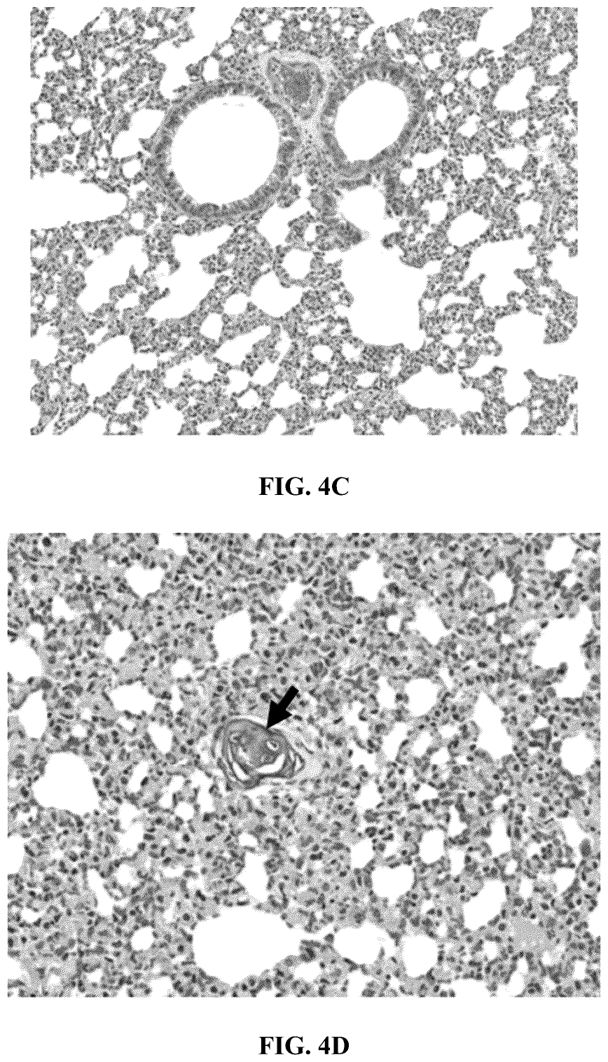

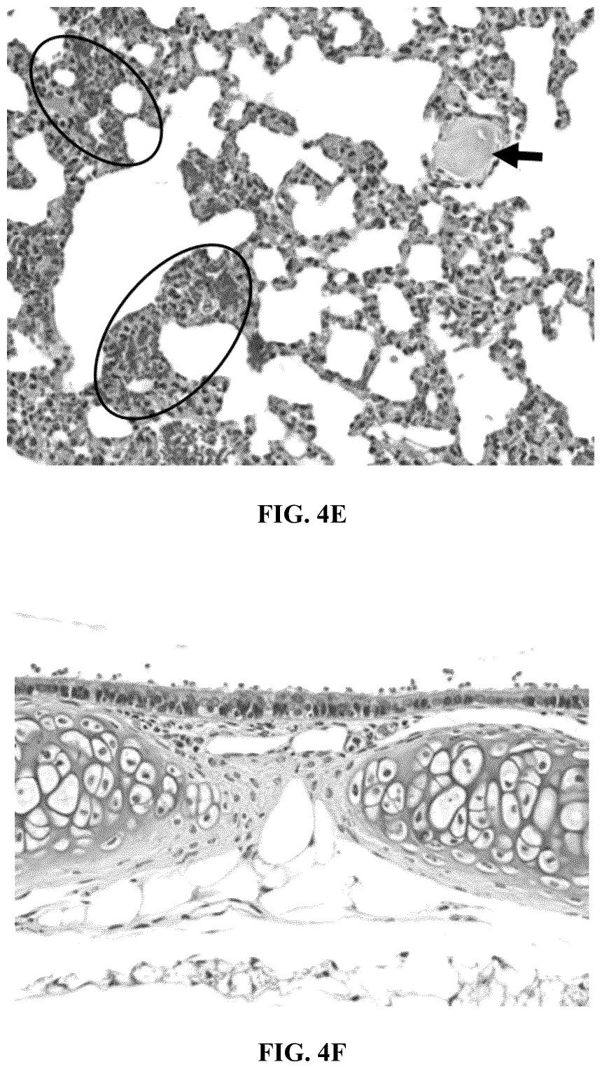

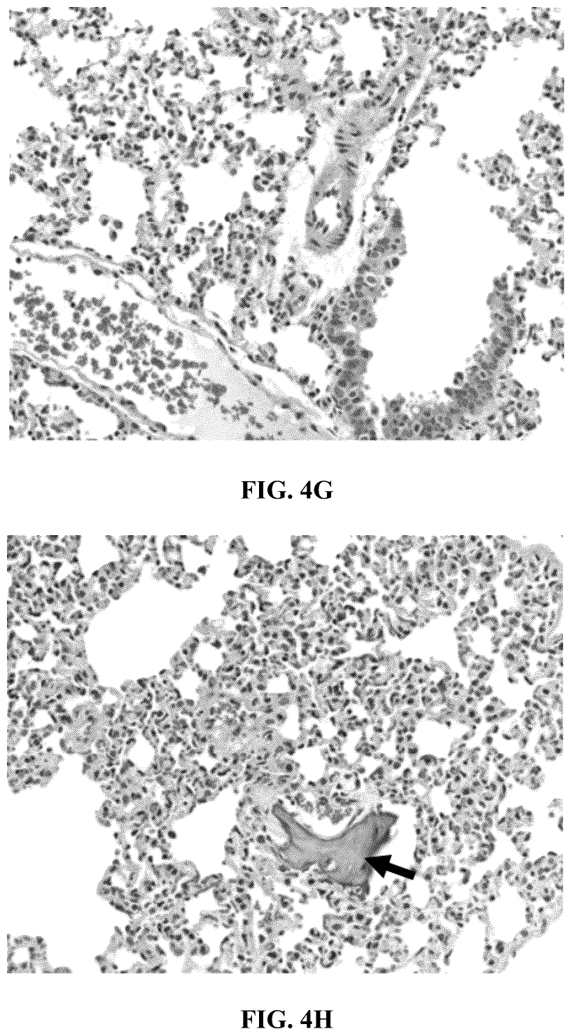

The results are shown in FIG. 4. No abnormality was found in the lung tissue of the group of hamsters that were not given medication (FIG. 4A to FIG. 4C). Of the six hamsters that were given medication (5 mg/ml) (FIG. 4D to FIG. 4I), however, three showed osseous metaplasia in alveoli (as indicated by the arrows in FIG. 4D, FIG. 4E, and FIG. 4H). It was suspected that the osseous metaplasia in alveoli was either idiopathic (i.e., not caused by the drug given) or a background condition (i.e., existing before drug administration) and therefore had nothing to do with the composition of the present invention. No abnormality was found in the lung tissue of the remaining hamsters that were given medication (FIG. 4F, FIG. 4G, and FIG. 4I).

### 4. Animal Challenge Study

The animal challenge study used hamsters as the animal model, and the hamsters were divided into the following groups:
1. Group without viral infection or medication (n=6)
2. Group with viral infection but without medication (n=6)
3. Group with viral infection and a low dose (1 mg/ml) of EIDD-1931 (n=6)
4. Group with viral infection and a high dose (5 mg/ml) of EIDD-1931 (n=6)

The drug, i.e., 1 mg/ml or 5 mg/ml EIDD-1931, was given at a dose of 50 μl by nasal dispensing in the 0.5^{th} hour, 8.5^{th} hour, 24.5^{th} hour, and 48.5^{th} hour after viral infection. The hamsters were sacrificed in the 72^{nd} hour after viral infection, and lung tissue was subsequently collected from the hamsters. The virus amount was determined by polymerase chain reaction (PCR), and biopsy sections were subjected to pathological examination in order to detect whether bronchiolar infiltration was accompanied by cellular inflammation or epithelial cell necrosis/degeneration.

Pathological changes attributable to the inoculation of SARS-CoV-2 include: (1) mixed cellular inflammation, peribronchial infiltration, and perivascular infiltration; (2) bronchial epithelial cell degeneration/necrosis accompanied or not accompanied by bronchiolar inflammatory infiltration; (3) alveolar wall necrosis; (4) vasculitis and vascular endothelialitis; and (5) substantial pulmonary hemorrhage and edema. The tissue examination aimed to detect the aforesaid pathological changes.

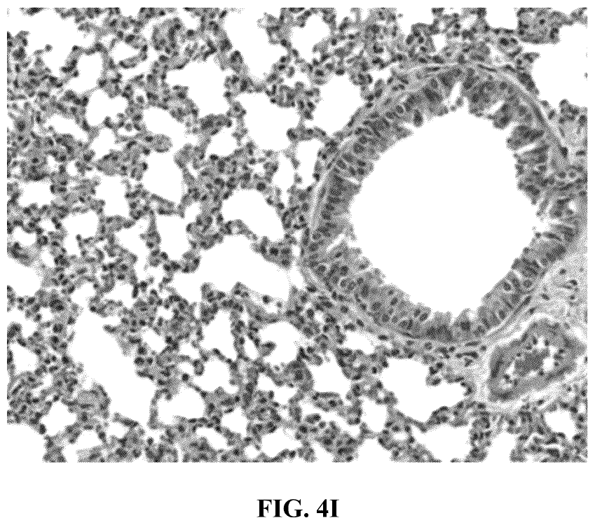

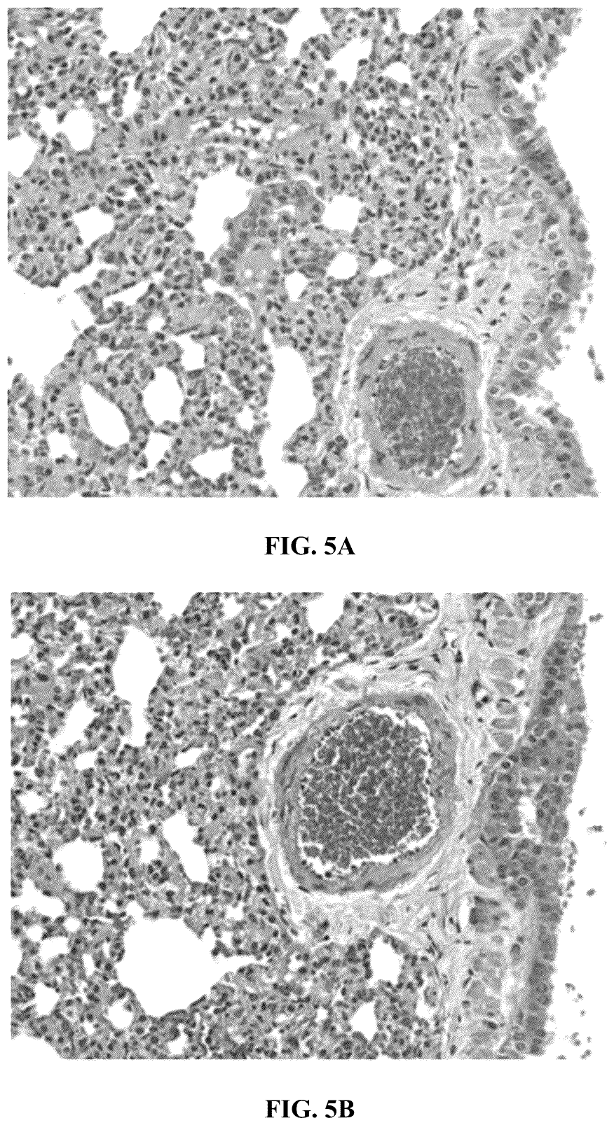

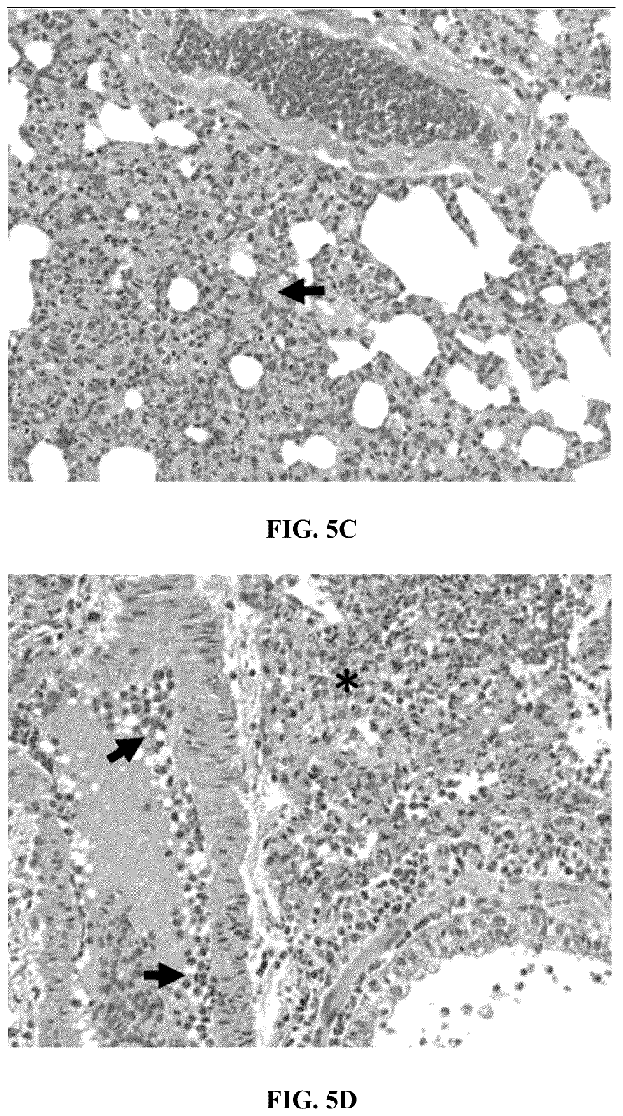

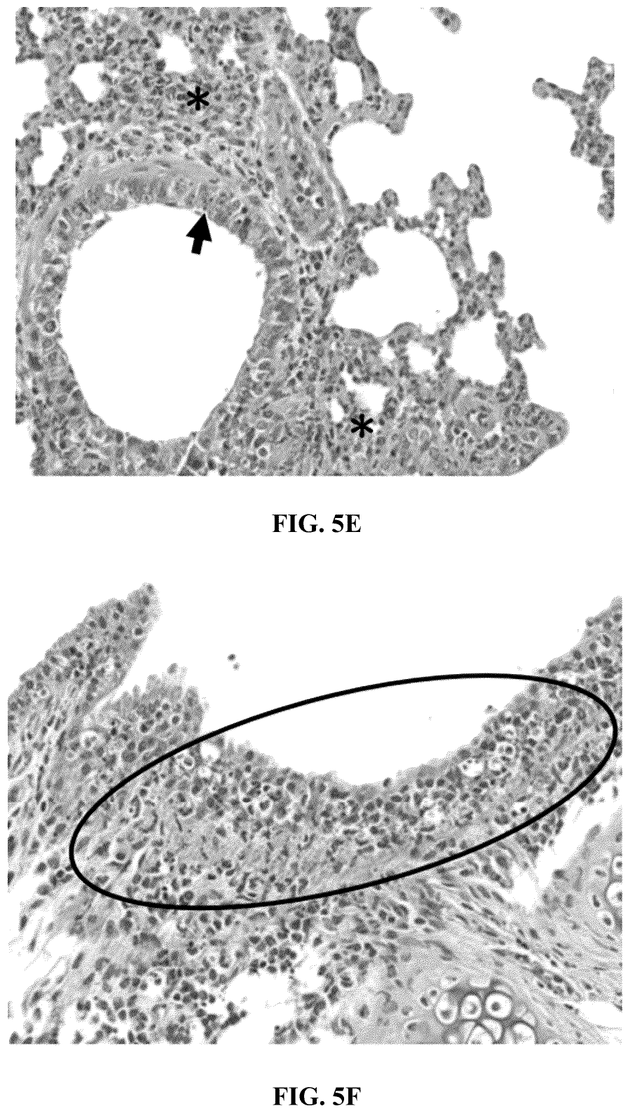

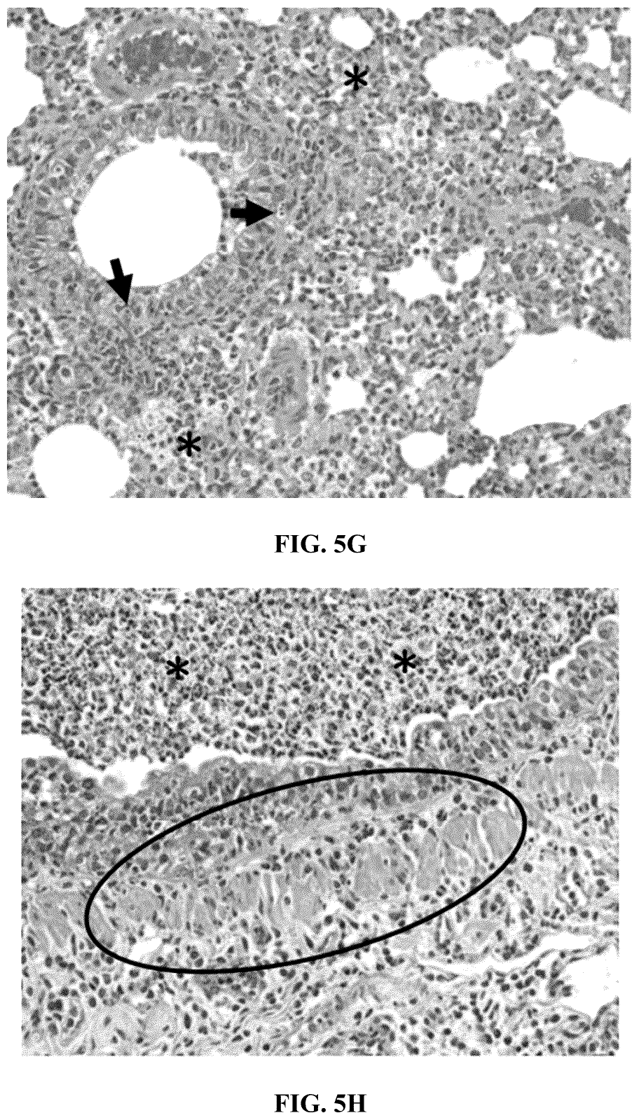

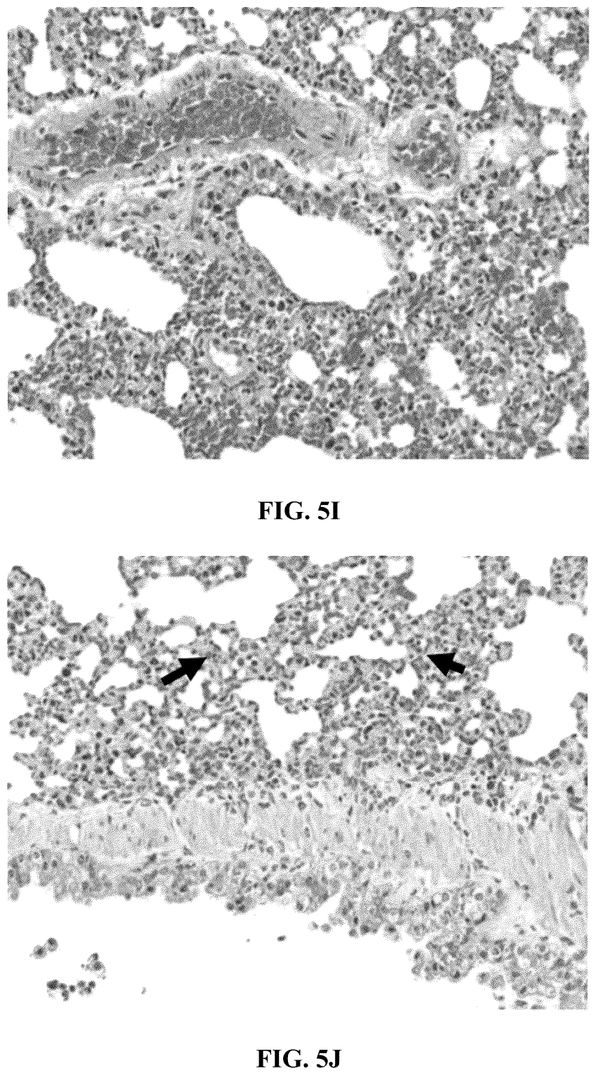

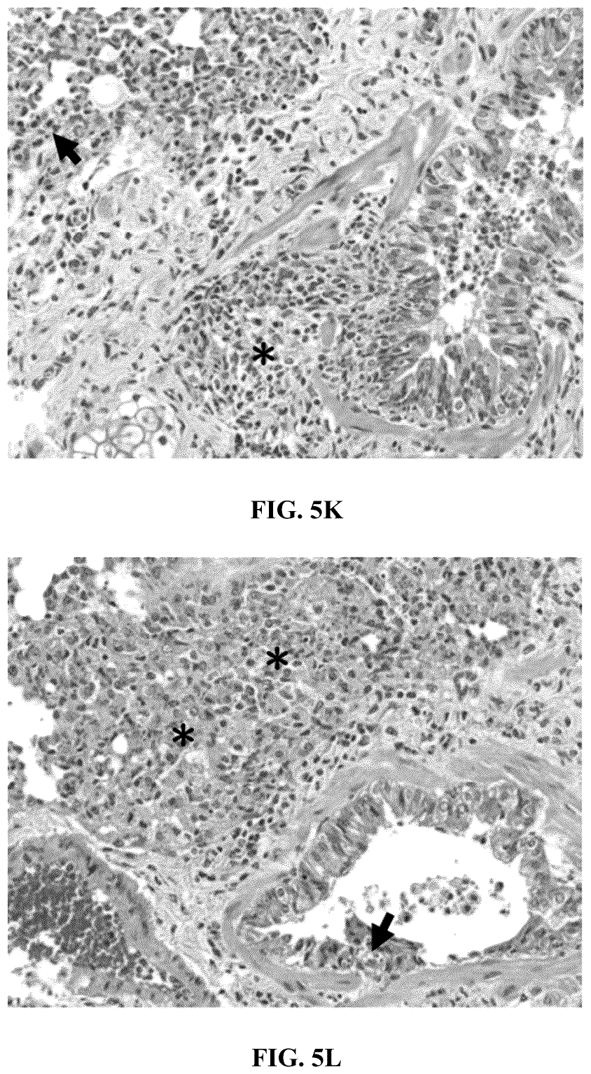

The results are shown in FIG. 5. Compared with the group with viral infection but without medication (FIG. 5D to FIG. 5F), the hamsters that were virus-infected and treated with either low-dose (FIG. 5G to FIG. 5I) or high-dose medication (FIG. 5J to FIG. 5L) had fewer instances of interstitial pulmonary inflammation and bronchiolar epithelial cell degeneration/necrosis. This indicates that the pharmaceutical solution composition of the present invention does have a therapeutic effect.

According to the above, the present invention provides a pharmaceutical solution composition that is highly stable and produces a relatively small amount of impurities. In addition, the pharmaceutical solution composition can be used in an oral mist inhaler so that a user can inhale the composition through the oral mist inhaler into the lungs, thereby not only allowing the composition to be rapidly absorbed, thanks to the capillaries covering the lungs, but also increasing the bioavailability of the drug significantly by reducing the metabolic process that the drug must go through in the user's body. Moreover, the particles formed by atomizing the composition are small, and the sprayed amount per spray can reach at least 13 mg. Compared with the conventional oral administration method, the oral mist inhaler of the invention is easier to use and can enhance the comfort and convenience of medicine consumption.

A detailed description of the present invention has been given above, and yet the foregoing description is based on only some preferred embodiments of the invention and is not intended to limit the scope of the invention. Any equivalent change or modification that is made according to the appended claims shall fall within the scope of the invention.

1. A pharmaceutical solution composition, comprising:
a therapeutically effective amount of molnupiravir or a metabolite thereof; and
a solvent.

2. The pharmaceutical solution composition of claim 1, wherein the metabolite of the molnupiravir is EIDD-1931 (β-D-N4-hydroxycytidine).

3. The pharmaceutical solution composition of claim 1, wherein the solvent is water.

4. The pharmaceutical solution composition of claim 1, further comprising a pharmaceutically acceptable excipient.

5. The pharmaceutical solution composition of claim 4, wherein the excipient is a preservative or a chelating agent.

6. The pharmaceutical solution composition of claim 5, wherein the preservative is benzalkonium chloride (BKC), benzethonium chloride, benzododecinium bromide, benzoic acid, benzyl alcohol, butylparaben, cetylpyridinium chloride (CPC), metacresol, methylparaben (MP), phenol, potassium sorbate, propylparaben, sodium borate, sorbic acid, or thimerosal, and the chelating agent is ethylenediaminetetraacetic acid (EDTA), edetate calcium disodium, anhydrous edetate calcium disodium, edetate disodium (EDTA-2Na), gluceptate sodium, or pentetic acid or a salt thereof.

7. The pharmaceutical solution composition of claim 1, wherein the molnupiravir or the metabolite thereof has a concentration of 2 mg/ml, 5 mg/ml, or 10 mg/ml.

8. The pharmaceutical solution composition of claim 1, wherein the pharmaceutical solution composition is administered by way of nasal drops, a nebulizer for oral or nasal inhalation, a nasal spray, a soft mist inhaler (SMI) for oral inhalation, or a metered-dose inhaler (MDI) for oral inhalation.

9. An oral mist inhaler, comprising:
a soft mist inhaler; and
the pharmaceutical solution composition of claim 1, wherein the soft mist inhaler is loaded with the pharmaceutical solution composition.

10. The oral mist inhaler of claim 9, wherein the pharmaceutical solution composition is atomized by the soft mist inhaler to form atomized particles, and an average sprayed amount of the atomized particles per spray is at least 13 mg.

11. The oral mist inhaler of claim 9, wherein the pharmaceutical solution composition is atomized by the soft mist inhaler to form atomized particles, and at least 50% of the atomized particles are smaller than 5.8 μm.

12. The oral mist inhaler of claim 11, wherein at least 60% of the atomized particles are smaller than 5.8 μm.
